(19) United States
(12) Patent Application Publication
(10) Pub. No.: US 2023/0402145 A1
(43) Pub. Date: Dec. 14, 2023

(54) NETWORK APPARATUSES AND METHODS FOR INTELLIGENT, REAL-TIME, PATIENT-CENTRIC OPIOID TREATMENT MANAGEMENT

(71) Applicant: OPOS, INC., San Mateo, CA (US)

(72) Inventors: Justin KROMELOW, Kirkland, WA (US); Rao MIKKILINENI, Cupertino, CA (US); Julie CAREY JOSEFSBERG, San Mateo, CA (US)

(21) Appl. No.: 18/130,029

(22) Filed: Apr. 3, 2023

Related U.S. Application Data
(60) Provisional application No. 63/352,212, filed on Jun. 14, 2022.

(51) Int. Cl. G16H 20/10; G16H 50/20

(52) U.S. Cl. CPC G16H 20/10; G16H 50/20

(57) ABSTRACT

The present invention relates a knowledge-based networked apparatus comprising at least one autopoietic node configured to monitor and maintain the stability of the knowledge network and maintain system availability, performance, security and regulation compliance, at least one cognitive node configured to manage the dynamics of opioid treatment process using various information processing knowledge structures that perform a collection of information from a variety of sources in a variety of forms, one or more functional nodes configured to provide algorithmic processing, machine learning and deep learning neural networks providing information processing of various domain specific functions; and wherein the at least one autopoietic node, at least one cognitive node and one or more functional nodes are configured to provide real-time actionable insights to optimize the opioid treatment according to the patient's condition by generating a common representation of the knowledge network with entities their relationships and behaviors.

### FIELD OF INVENTION

The present invention relates to systems and methods to monitor and manage opioid treatment for a patient in real-time using an integrated information processing platform.

### BACKGROUND

Opioids are widely used to manage chronic pain with almost 20 million people being prescribed a 30 day or longer prescription. According to the Center for Disease Control and Prevention (CDC), there are between 9 million and 18 million adults receiving opioids at a greater than recommended dosage. While opioids remain a significant and essential part of some patients' healing requirements, opioid use needs to be tailored to each individual according to their post-surgical pain threshold. A taper is a reduction in daily opioid dosage done to improve a patient's safety profile or quality of life. A successful taper plan reflects results in either a lower daily dosage or discontinuation of opioid therapy based on the patient's goals and risk profile. A taper should only be undertaken when it improves the patient's risk benefit profile, or when it is requested by the patient.

In order to determine a successful taper plan for a patient, health care providers must provide a complete assessment of risk benefit analysis (RBA) for continuing opioid therapy at the current dose, engage in shared decision making, formulate a patient-centered taper plan based on a reassessment of pain, functions and ongoing patient support.

Thus, building a customized taper plan and a thorough RBA requires a depth of knowledge about the patient's circumstances from various sources that need to be integrated in real-time to provide data-driven treatment to the patient.

Therefore, in light of the above discussion there is a need for a cognitive model of the opioid tapering therapy process and use it to provide real-time actionable insights to both the health care provider and the patient.

### OBJECTS OF THE INVENTION

The primary object of the present invention is to provide knowledge-based network apparatus configured to monitor patient function and activities of daily living and bridge the solution to monitor health across all common chronic disease co-morbidities.

Another object of the present invention is to automate the taper process in pain management treatment for providing recommendations by predicting the next tapering step ideal for the set goal or outcome for the patient in opioid reduction.

Yet another object of the present invention is to collaborate knowledge data from various domains and sources to create a cognitive model of the opioid tapering therapy process and use it to provide real-time actionable insights to both healthcare providers and patients.

### SUMMARY

The present invention relates to a knowledge-based networked apparatus comprising at least one autopoietic node configured to monitor and maintain the stability of the knowledge network and maintain system availability, performance, security and regulation compliance, at least one cognitive node configured to manage the dynamics of opioid treatment process using various information processing knowledge structures that perform a collection of information from a variety of sources in a variety of forms, one or more functional nodes configured to provide algorithmic processing, machine learning and deep learning neural networks providing information processing of various domain specific functions; and wherein the at least one autopoietic node, at least one cognitive node and one or more functional nodes are configured to provide real-time actionable insights to optimize the opioid treatment according to the patient's condition by generating a common representation of the knowledge network with entities their relationships and behaviors.

### BRIEF DESCRIPTION OF THE DRAWINGS

The disclosed embodiments have other advantages and features which will be more readily apparent from the following detailed description of the invention and the appended claims, when taken in conjunction with the accompanying drawings, in which:

FIG. 1 is an environment diagram of the knowledge-networked apparatus for pain management process, in accordance to an embodiment;

FIG. 2 illustrates an autopoietic network node in the knowledge network, according to an embodiment;

FIG. 3 illustrates a cognitive network node in the knowledge network, according to an embodiment;

FIG. 4 illustrates a knowledge network functional node of the networked system according to an embodiment;

FIG. 5 illustrates the hierarchical diagram 500 of the knowledge-based network apparatus 100, according to an embodiment;

FIG. 6 illustrates an operation flow diagram 600 of the knowledge-based network apparatus 100, according to an embodiment; and

FIG. 7 is a block diagram illustrating the mobile computing device 700, according to an example embodiment.

### DETAILED DESCRIPTION

The figures and the following description relate to preferred embodiments by way of illustration only. It should be noted from the following discussion that alternative embodiments of the structures and methods disclosed herein will be readily recognized as viable alternatives and may be employed without departing from the principles of what is claimed.

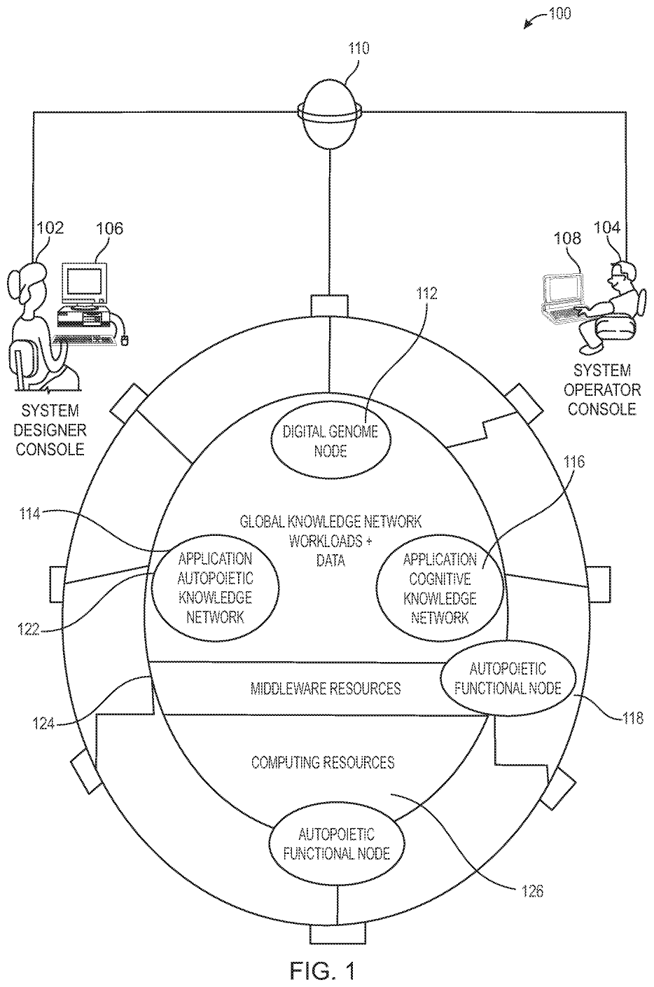

FIG. 1 is an environment diagram of a knowledge-based network apparatus 100 in relation to multiple system users 102, 104. The system users 102, 104 may have various roles in the networked system, and the users may use user devices 106, 108 respectively that may interact with a pain analytics server installed or accessed through the user devices 106, 108 over a network 110.

According to an embodiment, the network 110 may include, for example, one or more of the Internet, Wide Area Networks (WANs), Local Area Networks (LANs), analog or digital wired and wireless telephone networks (e.g. a public switched telephone network (PSTN), Integrated Services Digital Network (ISDN), a cellular network, and Digital Subscriber Line (DSL)), radio, television, cable, satellite, or any other delivery or tunneling mechanism for carrying data. Network 110 may include multiple networks or subnetworks, each of which may include, for example, a wired or wireless data pathway. The network 110 may include a circuit-switched network, a packet-switched data network, or any other network able to carry electronic communications (e.g., data or voice communications). For example, the network 110 may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. The network 110 may include one or more networks that include wireless data channels and wireless voice channels. The network 110 may be a wireless network, a broadband network, or a combination of networks including a wireless network and a broadband network.

According to an embodiment, the knowledge-based network apparatus 100 comprises of at least one knowledge network with one or more knowledge nodes configured to monitor a patient's function and activities of daily living and bridge the solution to monitor health across all common chronic disease co-morbidities. While knowledge-based network apparatus 100 is useful for a variety of pain management processes, it is primarily aimed at opioid treatment.

The knowledge-based network apparatus 100 can create one or more knowledge networks to perform one or more pain management tasks related to the care of a patient that may be variously not started, incomplete, or completed. The one or more knowledge networks are configured to specify, model, execute, monitor and manage various processes in real-time to synthesize information, assess and mitigate risk based on patient's objectives and best-known practices for managing the pain of a patient and delivering recommendations in real-time for adjusting the treatment plans of a patient. The input to the knowledge-based network apparatus 100 is data streams provided by various information sources of relevant information regarding the pain management treatment of a patient. The output is in the form of actionable insights provided to both the health-care provider and the patient.

The knowledge-based network apparatus 100 through one or more knowledge nodes of at least one knowledge network uses the opioid treatment management processes and the information sources to collect relevant data, uses the data to populate a model with a schema that models the dynamic processes and information flow involved in generating actionable insights. Each knowledge node performs certain tasks and communicates with other nodes that are related and participate in a collective behavior to designs an individualized and customized opioid reduction program with both pharmacological and psychological supports for the patients who are on opioids for reducing their pain.

Wherein the knowledge-based network apparatus 100 automates the process of taper treatment plans by gathering relevant information of the patient knowledge from various domains and sources to create a cognitive model of opioid tapering therapy process and uses it to provide real-time actionable insights to both the health-care provider and the patient. The cognitive model is deployed using the knowledge network. Knowledge data from various sources required by the health care provider and the patient are integrated in real-time to provide data-driven treatment to the patient and tapering plans are customized to the patient and adjusted in real-time based on patient's experience. The knowledge-based network apparatus 100 automates the taper process step recommendations by predicting the next tapering step for the patient based on the patient's monitored health time-line.

According to an embodiment, multiple knowledge networks can be stored in a digital genome 112 that can also be considered as a master knowledge repository. In one embodiment, the information of the digital genome can be accessed and stored on the pain analytics server. The digital genome 112 consists of various computational processes for processing information from various sources, in various forms in various distributed computing platforms with common knowledge data, wherein the knowledge data is represented in the form of knowledge structures.

The digital genome 112 specifies the knowledge network in the form of a network of networks (such as sub-networks) and executes the processes using a multi-tier architecture (e.g. a three-tier architecture), with various computing resources that are made available through distributed cloud providers or data centers or edge computing devices. The multi-tier architecture includes global knowledge network workloads and knowledge data as a primary layer 122, middleware resources as a secondary layer 124 and computing resources or edge devices as a tertiary layer 126.

A knowledge-based network apparatus 100 may refer to a structured representation for a patient's association with a domain of knowledge and the related communications or exchanges of the domain of knowledge of the paint management treatment such as opioid treatment. In an exemplary scenario, a knowledge network with the digital genome identifies eligible patients who are ready to reduce their opioid doses, regularly assesses patients throughout the reduction program, adapts the program to their individual needs and follows up throughout their reduction procedure. The knowledge network can grow and evolve with information collected during the opioid taper plan or treatment process.

In one embodiment, the information for the knowledge network can be stored in a set of dynamic database tables and/or searchable indexes. The set of dynamic database tables can be used to describe the knowledge networks, nodes, links among nodes, profiles of each patient (e.g., a set of databases to represent structured information and free text as unstructured information).

Knowledge data may refer to a collection of domain knowledge or information about the patients who are on opioids for reducing their pain. The information collected is relevant to patient goals, patient outcomes, patient risk factors, and patient tasks. The content of the knowledge data can include structured (e.g., attributes/hierarchical attributes), unstructured information (e.g. natural language or procedural codes), or other knowledge representations, such as logic and rules.

According to an embodiment, the various sources for collecting relevant information may include a plurality of record/logging systems such as hospital EHR systems, health information exchange EHR systems, clinical genetics/genomics systems, ambulatory clinic EHR systems, psychiatry/neurology EHR systems, insurance, collections or claims records systems, historical data for patient addition treatment, relapse information, other health services, claims data, apportionment data, related health services financial data and the like.

Further, the knowledge structures capture the knowledge data about various entities, their relationships and their behaviors when their properties change caused by internal or external events associated with patient data. The knowledge structures are dynamic in nature are configured to capture any changes in the work-flows of the opioid treatment plan or task for a patient when the information about the state of any entity associated with the patient changes.

The knowledge structures provide a common knowledge representation integrating the information obtained from various computations consisting of algorithmic processing, machine learning algorithms and deep learning neural networks.

The knowledge-based network apparatus 100 comprises at least one autopoietic node 114 configured to monitor and manage the knowledge network using distributed computing resources with location transparency to maintain end to end stability of the system to maintain availability, performance, security and regulatory compliance of information processing knowledge structures. Autopoiesis describes a technical system capable of regenerating, reproducing and maintaining itself by production, transformation and destruction of its components and the network of processes in these components.

Further, the knowledge-based network apparatus 100 comprises at least one cognitive node 116 configured to monitor and manage an integrated information model in the knowledge network. The cognitive node 116 integrates the results from various sources processed using various methods, such as processing of task-based work-flows of the opioid taper plan treatment, machine Learning to model the data to assess and manage risk profile for risk-based assessment and deep learning to identify patterns and relationships between various data from different sources in different forms associated with patient goal information relating to desired outcomes sought by the patient.

Further the knowledge-based network apparatus 100 consists of a plurality of functional nodes 118 configured information processing nodes that are connected with other networked nodes to exchange relevant information and collaborate in executing collective behaviors for determining the effective next step in the taper plan for a patient. While each node executes local tasks, the nodes that are connected together execute collaborative behaviors defined by the cognitive agent using the knowledge network. The purpose of the knowledge-based network apparatus 100 is to model the dynamic processes and information flows involved in opioid treatment, and provide real-time insights as the processes evolve.

In an exemplary scenario, a request may be submitted by a user of a user device to a pain analytics server for information on the next action in the opioid treatment. The client device may be a mobile device, such as telephone, PDA (Personal Digital Assistant), wireless handheld, etc. The request may also be sent by a user of an application that is external to the pain analytics server.

The pain analytics server interacts with the digital genome node 112 that interacts with the other networked knowledge nodes to collect the relevant information of the patient based on the set goals and outcomes and displays the determined recommendation or insight in real-time using text, voice or email communication, wherein the recommendations or insights may include but are not limited to a prescription drug monitoring program, medication interaction actions, scheduling a drug screening, viewing a patient report such as a trend report and the like. Further, the insights and/or recommendations are also notified to the health care providers and the patients.

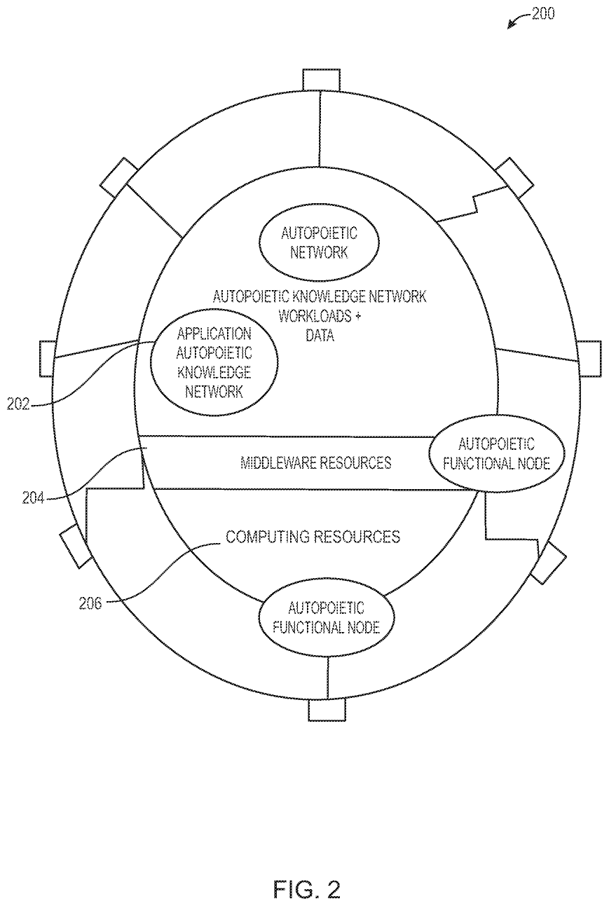

FIG. 2 illustrates an autopoietic network node 200 in the knowledge network, according to an embodiment. The autopoietic network node 200 is configured to identify and allocate the computing resources required to all the knowledge network functional nodes to execute their specified processes.

Further, each autopoietic network node 200 configures, monitors and manages the stability of the downstream knowledge network nodes executing various information processing functions for the knowledge-based network apparatus 100. Autopoiesis describes a technical system capable of regenerating, reproducing and maintaining itself by production, transformation and destruction of its components and the network of processes in these components.

The autopoietic network node 200 is further adapted to communicate with other nodes in a networked system and includes processing means located on selected nodes for controlling the nodes, the operation of the processing means maybe governed by a library of algorithms which includes a plurality of algorithms defining predetermined functions which mediate local and remote nodes of the networked system wherein the library of algorithms is stored in media readable by the processing means included within the pain analytics server in the networked system and distributed according to the functional requirements of at least part of the networked system. The autopoietic network node 200 may also include a plurality of sensing means adapted to sense parameters related to the patient's health in which the activities of the networked system through selection and execution of the algorithms are dependent on the satisfaction of set criteria of set outcomes and goals regarding the taper plan.

According, to an embodiment, the autopoietic network node 200 is in the form of a network of networks (such as sub-networks) and executes the processes using a multi-tier architecture (e.g., a three-tier architecture), with various computing resources that are made available through distributed cloud providers or data centers or edge computing devices. The multi-tier architecture includes autopoietic knowledge network workloads and knowledge data as a primary layer 202, middleware resources as a secondary layer 204 and computing resources or edge devices as a tertiary layer 206.

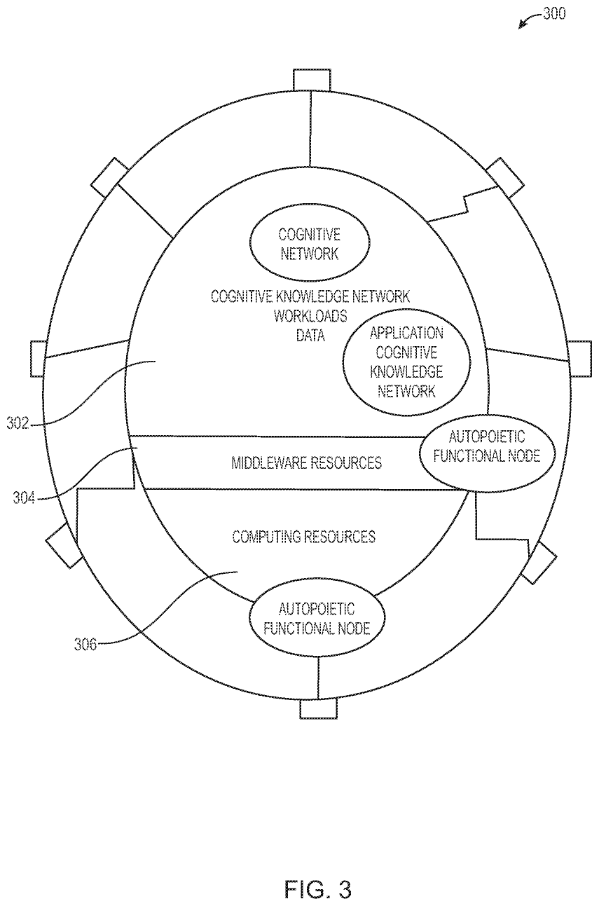

FIG. 3 illustrates a cognitive network node 300 in the knowledge network, according to an embodiment. The cognitive network node 300 is configured to monitor and manage computations in progress in the downstream knowledge network functional nodes to modify their evolution when required. Each cognitive node configures, monitors and manages the evolution of the downstream knowledge network nodes executing various information processing functions for the knowledge-based networked apparatus. The cognitive network node 300 is further adapted to communicate with nodes in a networked system, processing means is located on selected nodes for controlling the nodes, the operation of the processing means may be governed by a library of algorithms which includes a plurality of algorithms defining predetermined functions which mediate local and remote nodes of the networked system wherein the library of algorithms is stored in media readable by the processing means included within the pain analytics server in the networked system and distributed according to the functional requirements of at least part of the networked system.

According to an embodiment, the cognitive network node 300 is in the form of a network of networks (such as sub-networks) and executes the processes using a multi-tier architecture (e.g., a three-tier architecture), with various computing resources that are made available through distributed cloud providers or data centers or edge computing devices. The multi-tier architecture includes cognitive knowledge network workloads and knowledge data as a primary layer 302, middleware resources as a secondary layer 304 and computing resources or edge devices as a tertiary layer 306.

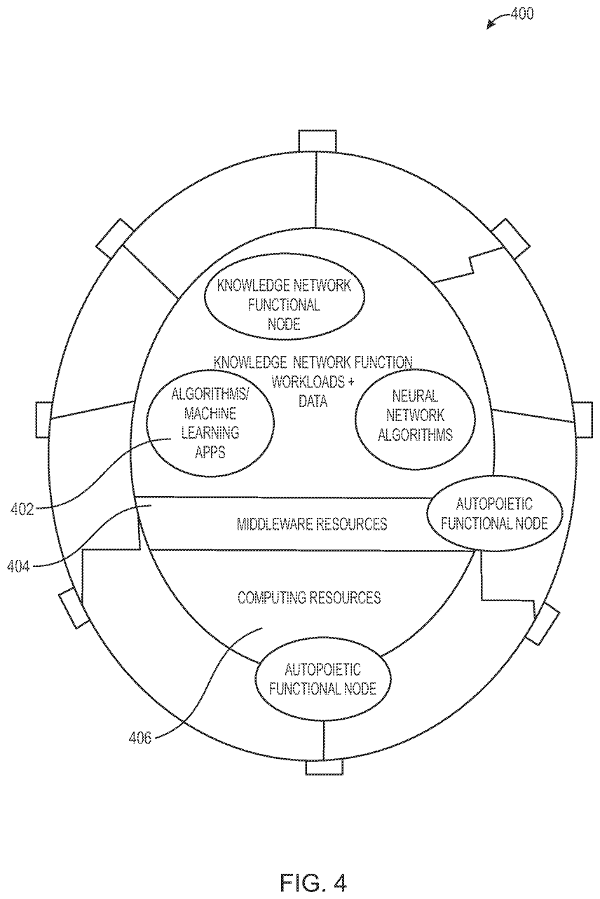

FIG. 4 illustrates a knowledge network functional node 400 of the networked system according to an embodiment. Each knowledge network functional node 400 is configured to provide algorithmic processing, machine learning and deep learning neural networks providing information processing of various domain-specific functions of the knowledge-based network apparatus 100. According to an embodiment, machine learning and deep learning neural networks of the knowledge network functional node 400 are capable of continual learning in a scalable manner to train the machine learning and deep learning neural networks models to attain acceptable performance on a new learning task while maintaining acceptable performance on earlier tasks.

According, to an embodiment, the knowledge network function node 400 is in the form of a network of networks (such as sub-networks) and executes the processes using a multi-tier architecture (e.g., a three-tier architecture), with various computing resources that are made available through distributed cloud providers or data centers or edge computing devices. The multi-tier architecture includes knowledge network function workloads and knowledge data as a primary layer 402, middleware resources as a secondary layer 404, and computing resources or edge devices as a tertiary layer 406.

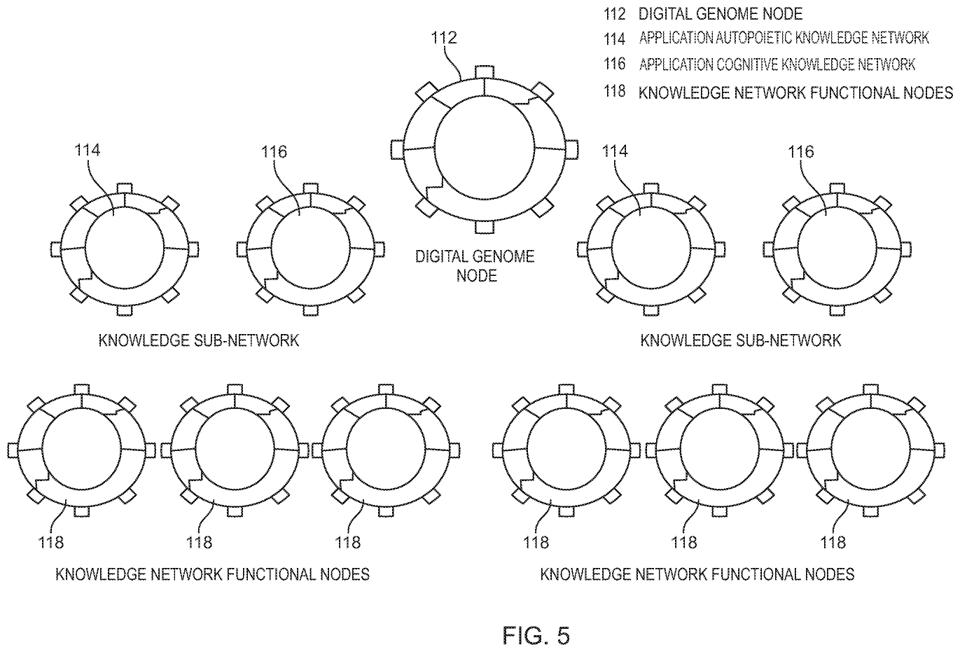

FIG. 5 illustrates the hierarchical diagram 500 of the knowledge-based network apparatus 100, according to an embodiment. According to an embodiment, the knowledge network 500 is represented by a node graph, where digital genome node 112 is connected to at least one cognitive knowledge node 116 and/or autopoietic knowledge node 114 and one or more functional nodes 118 wherein each node at least corresponds to the models managed by the digital genome node 112 of the knowledge-based network apparatus 100. In operation a link is added between two nodes when a knowledge-based interaction occurs between the two nodes. Examples of interactions include a user answering an opioid question, record any change in health parameters in a patient (such as, increase in blood pressure), etc.

Each knowledge sub-network includes a multi-layer structure, having an output layer generating an output of the sub-network and each node of the sub-network is successively trained to learn multiple tasks. Further each knowledge sub-networks are provided with algorithmic processing, machine learning and deep learning neural networks with various domain specific functions and data.

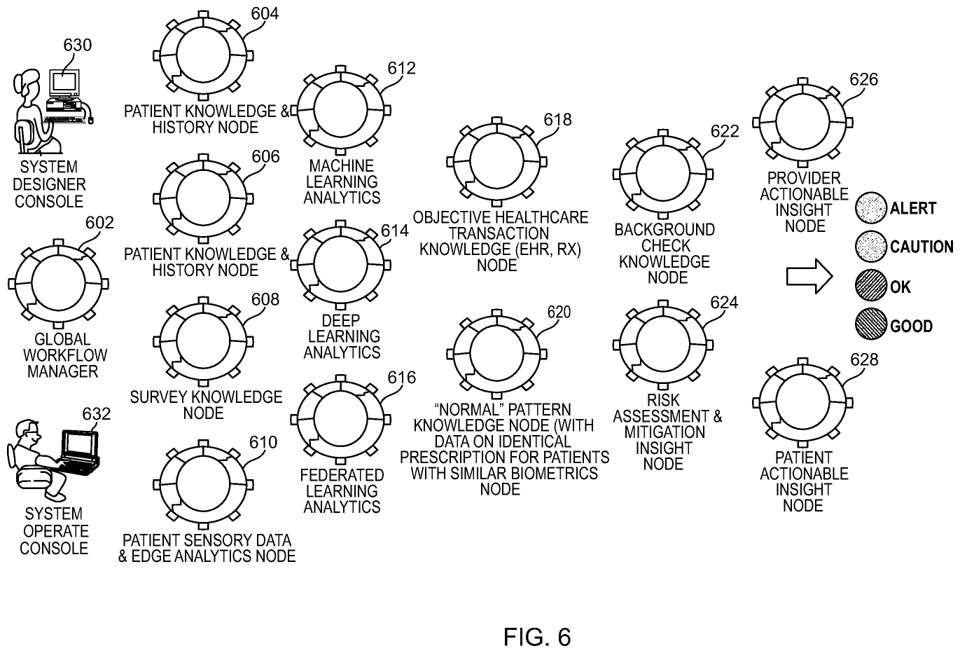

FIG. 6 illustrates an operation flow diagram 600 of the knowledge-based network apparatus 100, according to an embodiment. In an exemplary scenario, the knowledge network adopts knowledge data from various sources required by the health care provider and the patient. The results from each functional node and each sub-network are integrated in real time to provide data driven treatment to the patient and tapering plans are customized to the patient and adjusted in real time based on patient experience.

In the exemplary scenario, the knowledge nodes identified and classified can include global work flow manager 602, patient knowledge and history node 604, 606, a survey knowledge node 608, a patient sensory data & edge analytics node 610, machine learning analytics 612, deep learning analytics 614, federated learning analytics 616, objective healthcare transaction knowledge node 618, pattern knowledge node 620, background check knowledge node 622, risk assessment and mitigation insight node 624, provider actionable knowledge node 626, 628. Each of the nodes may be further accessed by system designer console 630 and system operator console 632.

Each sub-network is configured to collect relevant information of the patient, wherein the relevant information may be relevant to patient goals, patient outcomes, patient risk factors, and patient tasks collected and is used to optimize the taper plan based on the patient's condition and provide insights and/or recommendations. The recommendations may be used to identify the data that is pertinent for display on the user device. The insights and/or recommendations can include patient physical functioning, patient pain levels according to a suitable pain scale, patient activity levels, and outcomes sought by the patient. Each status of assessed outcome can be indicated through one or more colors, wherein a positive outcome can be indicated in green color, a warning in red color, medium levels of threat in yellow and orange colors respectively.

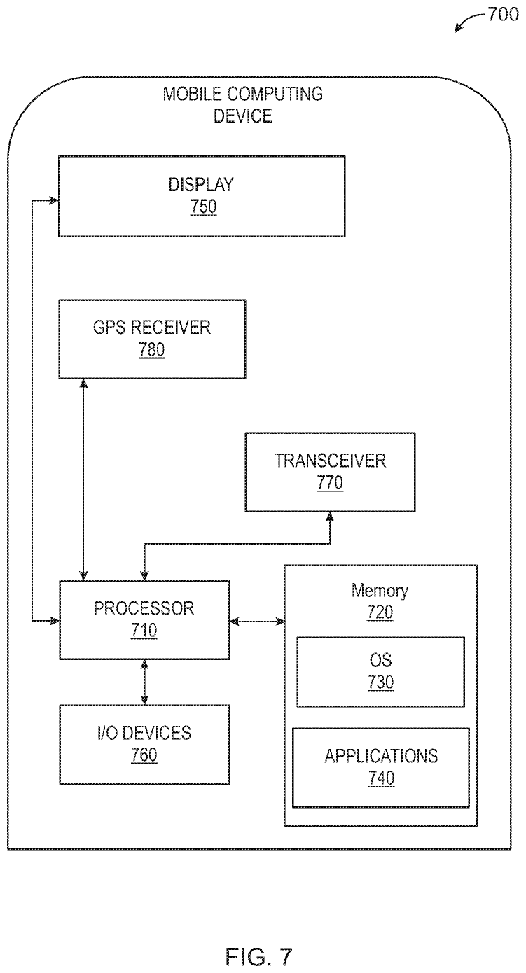

FIG. 7 is a block diagram illustrating the mobile computing device 700, according to an example embodiment. The device may correspond to, for example, one or more client machines or application servers. The mobile computing device 700 may include a processor 710. The processor 710 may be any of a variety of different types of commercially available processors suitable for mobile devices (for example, an XScale architecture microprocessor, a Microprocessor without Interlocked Pipeline Stages (MIPS) architecture processor, or another type of processor). A memory 720, such as a Random Access Memory (RAM), a Flash memory, or other type of memory, is typically accessible to the processor 710. The memory 720 may be adapted to store an operating system (OS) 730, as well as application programs 740, such as a mobile location enabled application that may provide location-based services to a user.

The processor 710 may be coupled, either directly or via appropriate intermediary hardware, to a display 750 and to one or more input/output (I/O) devices 760, such as a keypad, a touch panel sensor, a microphone, and the like. Similarly, in some embodiments, the processor 710 may be coupled to a transceiver 770 that interfaces with an antenna 790. The transceiver 770 may be configured to both transmit and receive cellular network signals, wireless data signals, or other types of signals via the antenna 790, depending on the nature of the mobile device 700. Further, in some configurations, a GPS receiver 780 may also make use of the antenna 790 to receive GPS signals.

Upon reading this disclosure, those of skill in the art will appreciate additional alternative structural and functional designs for a system and a process for automated identification and selection mechanism for candidates in a recruitment process through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A knowledge-based networked apparatus comprising:
at least one autopoietic node configured to monitor and maintain the stability of a knowledge network and maintain system availability, performance, security and regulation compliance;
at least one cognitive node configured to manage the dynamics of opioid treatment process using various information processing knowledge structures that perform a collection of information from a variety of sources in a variety of forms; and
one or more functional nodes configured to provide algorithmic processing, machine learning and deep learning neural networks providing information processing of various domain specific functions.

2. The apparatus as defined in claim 1, wherein the at least one autopoietic node, at least one cognitive node and one or more functional nodes are configured to provide real-time actionable insights to optimize the opioid treatment according to the patient's condition by generating a common representation of the knowledge network with entities their relationships and behaviors.

3. The apparatus as defined in claim 1, wherein the at least one cognitive node is configured to identify patterns and relationships between various data from different sources in different forms associated with patient goal information relating to desired outcomes sought by the patient.

4. The apparatus as defined in claim 1, wherein multiple knowledge networks are stored in a digital genome also considered as a master knowledge repository.

5. The apparatus as defined in claim 4, wherein the digital genome specifies the knowledge network in the form of a network of networks and executes a plurality of processes using a multi-tier architecture.

6. The apparatus as defined in claim 5, wherein the multi-tier architecture is configured to include:
a primary layer comprising global knowledge network workloads and knowledge data;
a secondary layer comprising a plurality of middleware resources; and
a tertiary layer comprising a plurality of computing resources or edge devices.

7. The apparatus as defined in claim 1, wherein the content of the knowledge data can include at least one of:
structured information or unstructured information or other knowledge representations, such as logic and rules.

8. The apparatus as defined in claim 1, wherein the knowledge network is further configured to:
identify eligible patients who are ready to reduce their opioid doses; and
assesses the identified patients through the reduction program.

9. The apparatus as defined in claim 1, wherein the knowledge network is configured to grow and evolve with information collected during the opioid taper plan or treatment process.

10. The apparatus as defined in claim 1, is further configured to perform the steps of:
receiving, by a processor, a request may be submitted by a user of a user device for information on the next action in the opioid treatment;
interacting, by the processor, with the digital genome node that interacts with the other networked knowledge nodes to collect the relevant information of the patient based on the set goals and outcomes; and
displaying, by the processor, the determined recommendation or insight in real-time using text, voice or email communication on the user device.

11. A method of using knowledge-based networked apparatus, the method comprising:
monitoring and maintaining the stability of a knowledge network and maintain system availability, performance, security, and regulation compliance using at least one autopoietic node;
managing the dynamics of opioid treatment process using various information processing knowledge structures that perform a collection of information from a variety of sources in a variety of forms using at least one cognitive node; and
providing algorithmic processing, machine learning and deep learning neural networks providing information processing of various domain specific functions using one or more functional nodes.

12. The method as defined in claim 11, wherein the at least one autopoietic node, at least one cognitive node and one or more functional nodes are configured to provide real-time actionable insights to optimize the opioid treatment according to the patient's condition by generating a common representation of the knowledge network with entities their relationships and behaviors.

13. The method as defined in claim 11, wherein the at least one cognitive node is configured to identify patterns and relationships between various data from different sources in different forms associated with patient goal information relating to desired outcomes sought by the patient.

14. The method as defined in claim 11, wherein multiple knowledge networks are stored in a digital genome also considered as a master knowledge repository.

15. The method as defined in claim 14, wherein the digital genome specifies the knowledge network in the form of a network of networks and executes a plurality of processes using a multi-tier architecture.

16. The method as defined in claim 14, wherein the multi-tier architecture is configured to include:
a primary layer comprising global knowledge network workloads and knowledge data;
a secondary layer comprising a plurality of middleware resources; and
a tertiary layer comprising a plurality of computing resources or edge devices.

17. The method as defined in claim 11, wherein the content of the knowledge data can include at least one of: structured information or unstructured information or other knowledge representations, such as logic and rules.

18. The method as defined in claim 11, wherein the knowledge network is further configured to:
identify eligible patients who are ready to reduce their opioid doses; and
assesses the identified patients through the reduction program;

19. The method as defined in claim 11, wherein the knowledge network is configured to grow and evolve with information collected during the opioid taper plan or treatment process.

20. The method as defined in claim 11, is further configured to perform the steps of:
receiving, by a processor, a request may be submitted by a user of a user device for information on the next action in the opioid treatment;
interacting, by the processor, with the digital genome node that interacts with the other networked knowledge nodes to collect the relevant information of the patient based on the set goals and outcomes; and
displaying, by the processor, the determined recommendation or insight in real-time using text, voice or email communication on the user device.
